(19) United States
(12) Patent Application Publication
(10) Pub. No.: US 2007/0191685 A1
(43) Pub. Date: Aug. 16, 2007

(54) Top handled surgical clamp

(75) Inventors: Steven LeVahn, Lino Lakes, MN (US); Ronald Von Wald, Centerville, MN (US); Todd W. Sharratt, Stillwater, MN (US); Steven Karl Jacobson, St. Paul, MN (US)

Correspondence Address: SHEWCHUK IP SERVICES, 533 77TH STREET WEST, EAGAN, MN, 55121, US

(73) Assignee: LeVahn Intellectual Property Holding Company, LLC, St. Paul, MN (US)

(21) Appl. No.: 11/352,816

(22) Filed: Feb. 13, 2006

(51) Int. Cl. A61B 1/32

(52) U.S. Cl. 600/227

(57) ABSTRACT

A surgical clamp includes a support frame clamp member, a retraction shaft clamp member and a handle linked to the support frame clamp member and the retraction shaft clamp member for moving the clamp members between loosened and tightened positions. Both the support frame clamp member and the retraction shaft clamp member permit transverse attachment. The shaft clamp and the support frame permit pivoting of the retraction shaft relative to the support frame. The handle is always in alignment with the retractor shaft, with a gripping portion extending above and in line with the shaft but attached to the clamp below the shaft. With this clamp and handle configuration, the clamp maintains a very low profile while still allowing easy access to the handle from above during the tightening and/or loosening motions, permitting a single handed “scissors action” tightening.

### CROSS-REFERENCE TO RELATED APPLICATION(S)

None.

### BACKGROUND OF THE INVENTION

The present invention relates to the field of surgical tools, and particularly to the design and manufacture of surgical retractor systems. Surgical retractor systems are used during surgery to bias and hold tissue in a desired position. As one example, some surgical procedures require anterior access to the spine, through the patient's abdomen. Tissue such as skin, muscle, fatty tissue and interior organs needs to be held retracted to the side so the surgeon can obtain better access to the vertebrae structures of primary interest.

Surgical retraction may be performed by one or more aides using handheld tools, with the most basic retractor apparatus being a tongue depressor. More commonly now in sophisticated operating rooms during abdominal or chest surgery, a surgical retractor system or assembly is used. The retractor assembly may, for instance, include a ring or support frame which is rigidly supported from the patient's bed above and around the surgical incision location, with a number of clamps and retractor blades to hold back tissue proximate to the surgical incision. Other retraction systems, such as those disclosed in U.S. Pat. Nos. 6,315,718, 6,368,271 and 6,659,944 to Sharratt, incorporated herein by reference, may not include a ring and/or may be directed at other types of surgery. Clamps may also be used to attach the ring or support frame to a support post and/or part of the bed frame.

In devising a proper clamping structure, the clamp should give the surgeon flexibility in quickly assembling the retraction system and in placement of the various retractors. (The term “surgeon” is used herein as including the person operating the clamp, who may or may not be the person performing the actual surgery.) Once the various retractors are in place and oriented and pulled as desired, the retraction system clamps should allow quick and easy tightening so the entire retraction system is maintained fixedly in place. Once tightened the retraction system should be unobtrusive so neither the tissue held retracted nor the retraction system interfere in any way with the surgeon or the surgical procedure. After surgery is completed (or perhaps once or more during surgery), the retraction system should quickly loosen and/or disassemble so as relax the retracted tissue and minimize damage to the retracted tissue. Surgical retractor systems must be robust and strong, as even a slight possibility of failure during use is not tolerated. Surgical retractor assemblies should be readily reusable, including sterilizable, for use in multiple surgeries. Surgical retractor systems should maintain a relatively low cost. Improvements in surgical retractor clamps and systems can be made in keeping with these goals.

### BRIEF SUMMARY OF THE INVENTION

The present invention is a surgical clamp and clamping system having two clamp openings, one for a support frame and another for a retractor or tool shaft. Both of these openings permit transverse attachment. A handle tightens the clamp. The handle attaches to the clamp at a location lower than the shaft, while at the same time having a gripping portion which extends above the shaft axis. With this clamp and handle configuration, the clamp maintains a very low profile while still allowing easy access to the handle from above during the tightening and/or loosening motions. In one aspect, the handle maintains alignment with the shaft during pivoting of the shaft relative to the support frame, permitting a single handed “scissors action” tightening.

### BRIEF DESCRIPTION OF THE DRAWINGS

FIG. 1 is a perspective view of a prior art surgical retractor system showing clamp location and use during surgery.

FIG. 2 is a perspective view of a preferred surgical clamp in accordance with the present invention.

FIG. 3 is an exploded perspective view of the clamp of FIG. 2.

FIG. 4 is an elevational side view of the clamp of FIGS. 2 and 3, showing the loosened position of the handle in dotted lines.

FIG. 5 is a plan view of the clamp of FIGS. 2-4, showing the range of adjustment of the shaft and handle relative to the support frame.

FIGS. 6 and 7 are end views of the clamp of FIGS. 2-5.

FIG. 8 is an opposing elevational side view of the clamp of FIGS. 2-7 in the loosened position.

FIG. 9 is a side view of the clamp of FIG. 8 in the tightened position.

FIG. 10 is a perspective view of an alternative surgical clamp in accordance with the present invention.

While the above-identified drawing figures set forth preferred embodiments, other embodiments of the present invention are also contemplated, some of which are noted in the discussion. In all cases, this disclosure presents the illustrated embodiments of the present invention by way of representation and not limitation. Numerous other minor modifications and embodiments can be devised by those skilled in the art which fall within the scope and spirit of the principles of this invention.

### DETAILED DESCRIPTION

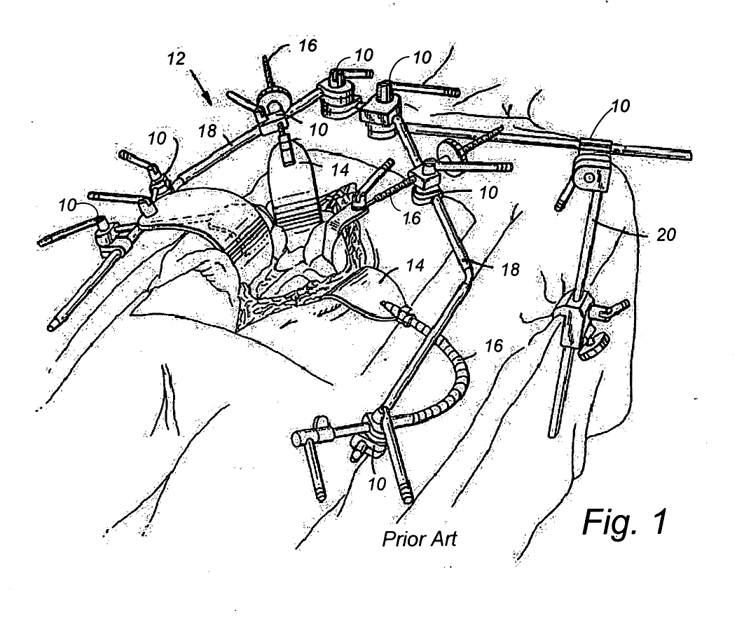

As shown in FIG. 1, one or more prior art clamps 10 are used in a surgical retraction system 12. The surgical retraction system 12 is used to support several surgical retraction blades 14 during surgery. The retractor blades 14 may be in accordance with prior art systems, or in accordance with those disclosed in pending application Ser. No. 11/247,817 filed Oct. 11, 2005, assigned to the assignee of the present invention and incorporated by reference. Each retractor blade 14 is attached to a retractor blade shaft 16. Each retractor blade shaft 16 is clamped with a clamp 10 to a retractor ring or support frame 18. The clamps 10 permit the surgeon to adjust the horizontal location (in and out) as well as the angular orientation of the shafts 16 and then permit the surgeon to securely fasten each retractor blade 14 once a desired position and orientation is achieved. The retractor ring 18 is supported relative to the patient's bed, such as by clamping to one or more retractor posts 20, and one or more clamps 10 can be used in this location as well. Each of the retractor blade shaft 16, the retractor ring 18 and the retractor post 20 may be formed primarily of a metal material such as surgical stainless steel as known in the surgical retractor art. In addition to the retractor components shown, clamps 10 can also or alternatively be used to clamp surgical tools or other apparatus (not shown), such as a camera, a light or a catheter to a support structure or together.

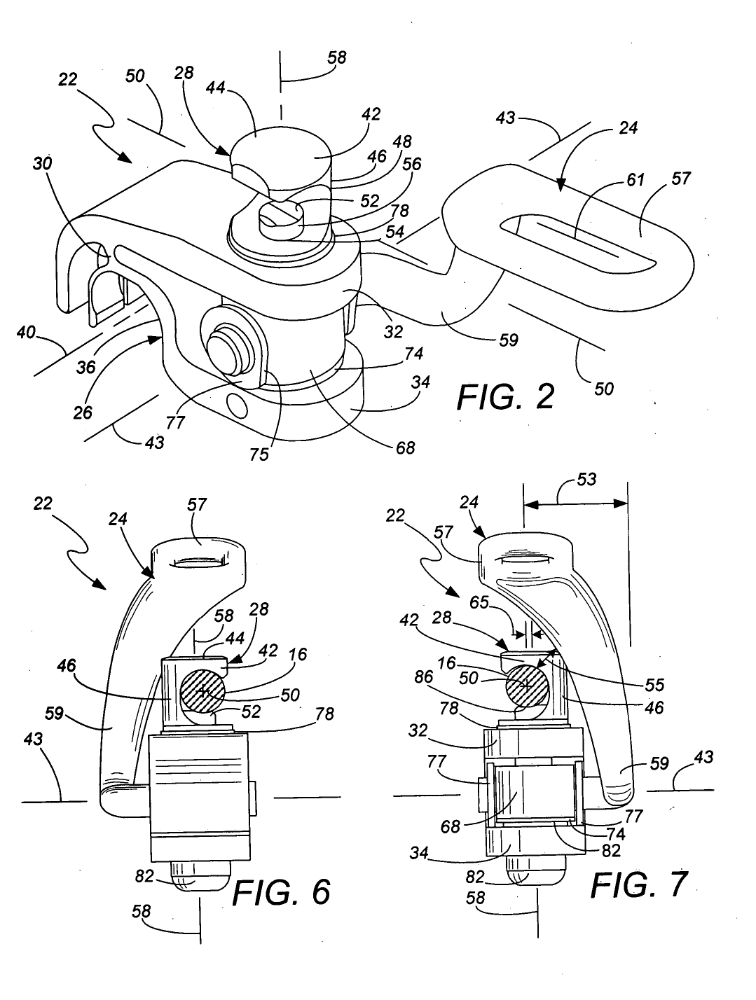

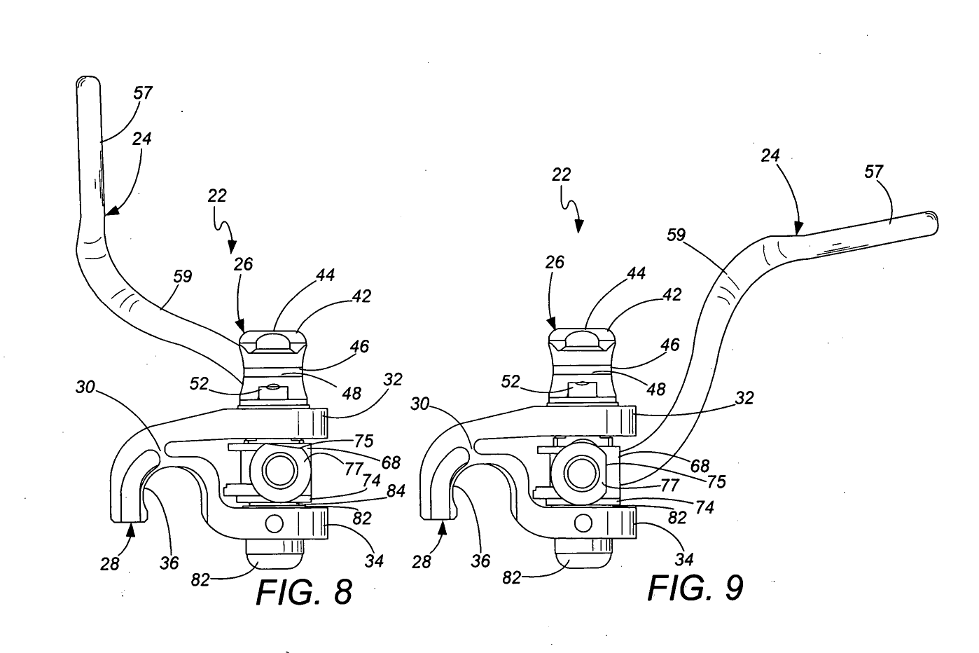

The clamp 22 of the present invention as shown in FIGS. 2-9 can be used in place of any or all of the prior art clamps 10. The clamp 22 primarily includes a tightening handle 24, a first clamp member 26 (in the lower position as shown in FIGS. 2-9, it being recognized that orientation of the clamp 22 may depend upon use as shown in FIG. 1) and a second clamp member 28 (in the upper position as shown in FIGS. 2-9). For ease of description, the first or lower clamp member 26 will be called a “frame” clamp and the second or upper clamp member 28 will be called a “shaft” clamp, recognizing that the first clamp 26 may attach to a rod other than the support frame 18 and the second clamp 28 may attach to a rod other than a retractor shaft 16.

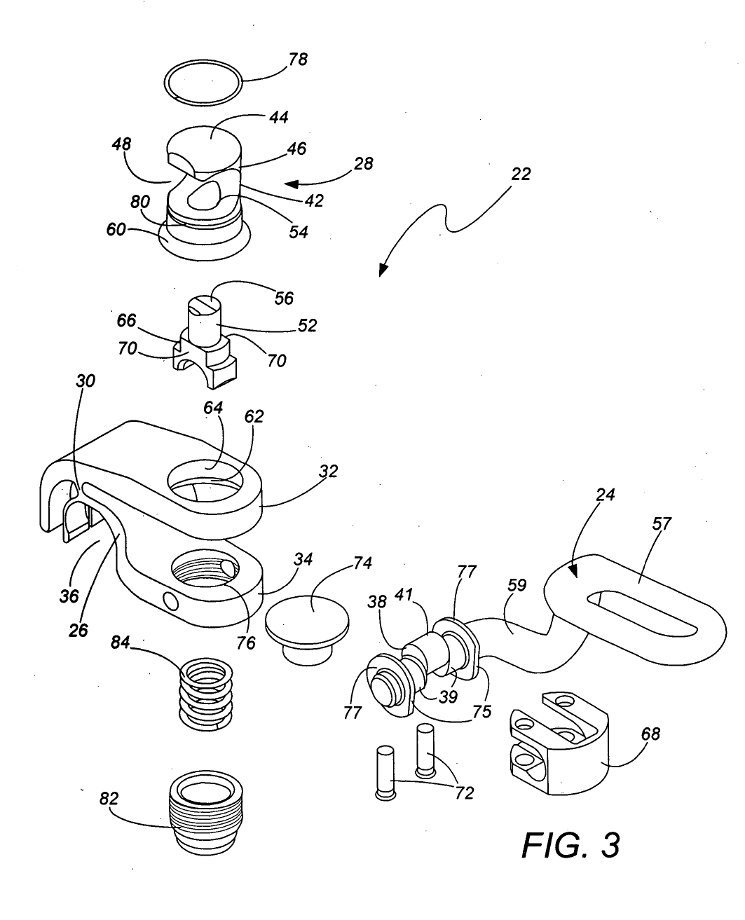

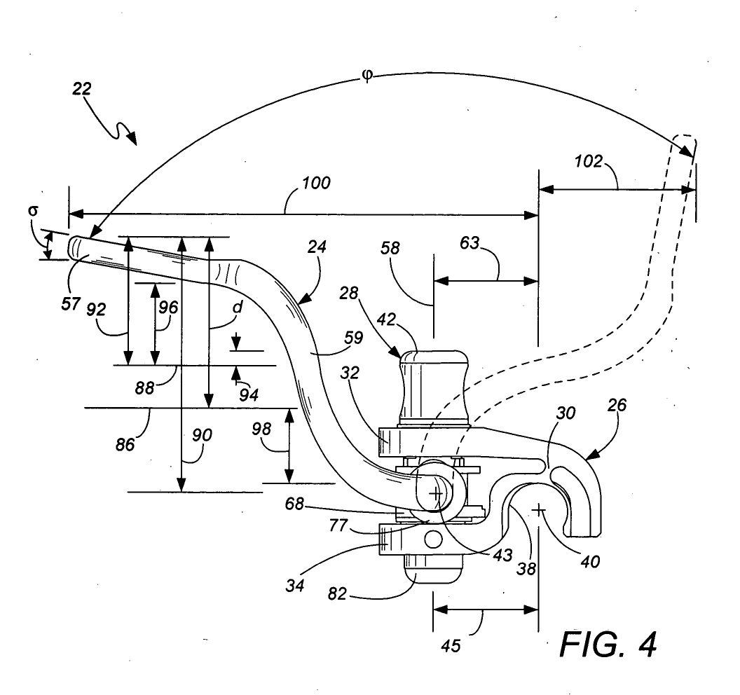

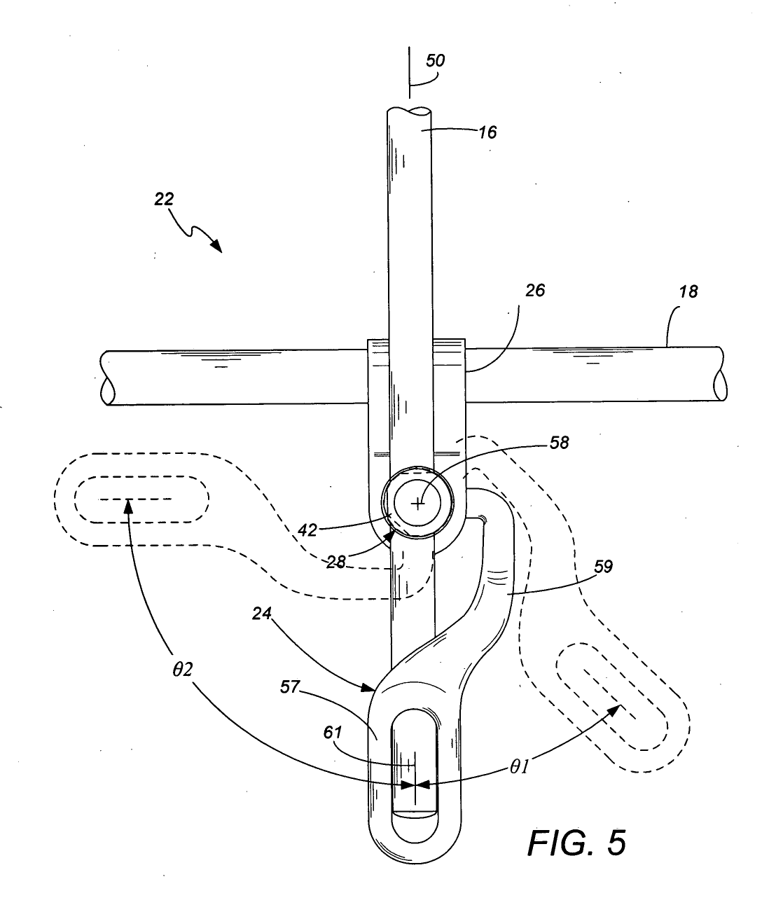

The frame clamp 26 may be a fulcrum clamp as generally disclosed in U.S. Pat. No. 5,727,899 and in application Ser. No. 10/664,195 filed Sep. 17, 2003 and Ser. No. 11/330,625 filed Jan. 12, 2006, all incorporated by reference. The preferred fulcrum clamp 26 thus includes a fulcrum portion 30 extending between an upper leg portion 32 and a lower leg portion 34. The fulcrum portion 30 allows the size of the frame clamp opening 36 to change based upon biasing the upper leg portion 32 away from the lower leg portion 34. A wedge or cam 38 (shown in FIG. 3) positioned between the upper and lower leg portions 32, 34 is movable to force the upper and lower leg portions 32, 34 apart and causes the fulcrum portion 30 to flex. As the fulcrum portion 30 flexes, the frame clamp opening 36 constricts. When the frame clamp opening 36 constricts, the clamp 22 can frictionally attach onto the support frame 18. The frame clamp opening 36 extends longitudinally on the frame 18 for a sufficient distance to define a rod axis 40 (shown in FIGS. 2 and 4) and orientation of the frame 18 (FIG. 5) in the frame clamp opening 36. In the preferred embodiment, the frame clamp 26 is about ¾ inch wide.

A significant advantage of using a fulcrum clamp for the frame clamp 26 is that it includes a clamping opening 36 which is open from below. The clamping opening 36 therefore permits the frame clamp 26 to be placed on the retractor support frame 18 transversely, i.e., without requiring longitudinal threading of the clamp body onto the support frame 18 and without moving any other surgical equipment that has been previously disposed upon the retractor support frame 18. The frame clamp 26 may be lightly and quickly snapped onto the support frame 18 at any substantially straight location desired by the surgeon. The frame clamp opening 36 is sized to mate with the cross-sectional size and shape of the support frame 18, such as a ½″ diameter cylindrical shape. In the loosened position, the frame clamp 26 permits substantially unimpeded longitudinal movement of the clamp 22 on any linear portion of the support frame 18, as well as substantially free rotation of the clamp 22 about the support frame axis 40.

Another significant advantage of using a fulcrum clamp is that the clamping force is easily made to be self-sustaining by use of a wedge or cam member 38 placing equal and opposite forces on the upper and lower leg portions 32, 34. Once the frame clamp 26 is closed to a tightened position, it does not require further application of force or holding by the surgical staff to remain in the tightened position. The preferred frame clamp cam 38 includes two outer lobes 39 for pushing downward and a central lobe 41 for pushing upward so it can provide a balanced force and for ease of manufacture and assembly. Many other types of mechanical wedges could alternatively be used for providing and maintaining the clamping force.

A third significant advantage of using a fulcrum clamp is that the actuating mechanism can be positioned closely in to the support frame 18, and at the same elevation as the support frame 18. This is best shown with reference to FIG. 4, wherein the handle 24 attaches closely in to the frame axis 40. In the preferred embodiment, the handle 24 and cam 38 pivots about a horizontal axis 43 which has an offset 45 of only about ⅞^{th} of an inch from the frame axis 40. As will be appreciated throughout this discussion, the location of the handle 24 for the frame clamp 26 relative to the support frame 18 and relative to the retractor shaft 16 provides significant benefits during surgery.

The shaft clamp 28 is preferably also open for side or top attachment of the retractor shaft 16, such as the side attachment clamp shown. As best shown in FIGS. 2, 3, 6 and 7, the shaft clamp 28 primarily includes a stanchion head 42 having a top stanchion 44 extending rigidly from a side stanchion 46. The inside surfaces of the top stanchion 44 and side stanchion 46 are sized and shaped to mate with the retractor shaft 16. The shaft clamp opening 48 extends longitudinally on the shaft 16 for a sufficient distance to define a rod axis 50 and orientation of the shaft 16 in the shaft clamp opening 48. The shaft clamp opening 48 also defines the placement of the bottom and top surfaces 86, 88 of the shaft 16. In the preferred embodiment, the shaft clamp 28 is about ½ inch wide.

The shaft clamp 28 is preferably activated by the same handle 24 as the frame clamp 26. To achieve the simultaneous tightening with a single handle 24, pivoting movement of the handle 24 not only causes the wedge 38 to increase separation between the upper and lower legs 32, 34, but also moves a pin 52 vertically upward to press the retractor shaft 16 against the top stanchion 44. The pin 52 translates or slides in a pin bore 54 in the bottom of the shaft clamp 28. The pin bore 54 intersects the shaft clamp opening 48, so the pin 52 can be biased against the outer profile of the shaft 16 by sliding the pin 52 in the pin bore 54.

A significant advantage of using a side snap clamp for the shaft clamp 28 is that it includes a clamping opening 48 which is open from above the support frame 18. The clamping opening 48 therefore permits the retractor shaft 16 to be placed into the side snap clamp 28 transversely, i.e., without requiring longitudinal threading of the shaft 16 through the shaft clamp opening 48. The handle 24 is preferably oriented to the side with a horizontal offset 53 providing a minimum clearance 55 (best shown in FIG. 7) relative to the retractor shaft 16 to further facilitate access during surgery to quickly and easily snap the shaft 16 into the clamping opening 48. With the preferred handle 24, the horizontal offset 53 is about 0.8 inches, and the minimum clearance 55 is about 0.2 inches.

In the preferred embodiment, the handle 24 includes a grasping portion 57 and an arm portion 59 extending from the cam 38. The grasping portion 57 is centered relative to the shaft axis 50, so biasing the handle 24 tighter or looser by the surgeon will result in no net rotational moment of the clamp 22 about the shaft axis 50. In the preferred embodiment as shown in FIG. 5, the grasping portion 57 defines a grasping axis 61 vertically in line with the shaft axis 50.

The shaft 16 may be lightly and quickly snapped into the shaft clamp 28 at any longitudinal location and angular orientation (i.e. either pivoted about the support frame 18 to a non-horizontal angle, or pivoted about a vertical axis 58 as described below) desired by the surgeon. The shaft clamp opening 48 is sized to mate with the cross-sectional size and shape of the shaft 16, such as a ⅜″ diameter cylindrical shape. In the loosened position, the shaft clamp 28 permits substantially unimpeded longitudinal movement of the shaft 16 in the shaft clamp opening 48, as well as substantially free rotation of the shaft 16 about its axis 50 in the shaft clamp opening 48.

The shaft clamp 28 is pivotable relative to the frame clamp 26 about the vertical axis 58. To achieve the pivoting feature, the shaft clamp 28 is attached to the frame clamp 26 through a rotatable attachment. After the clamp 22 is positioned on the support frame 18 and the shaft 16 is positioned in the shaft clamp 28 but before the handle 24 is moved from the loosened position to the tightened position, the shaft 16 is pivotable about the pivot axis 58. As shown in FIG. 4, an offset 63 between the frame axis 40 and the pivot axis 58 for the shaft 16 is as small as possible, e.g., axis 40 and axis 58 would intersect in the ideal design. In the preferred embodiment, the vertical pivot axis 58 for the shaft clamp 28 intersects the horizontal pivot axis 43 for the handle 24 and cam 38, and thus has an offset 63 from the frame opening axis 40 of only about ⅞^{th} of an inch.

As best shown in FIG. 5, the pivot axis 58 for the shaft 16 is preferably centered to bisect the frame clamp 28. As best shown in FIG. 7, an offset 65 between the pivot axis 58 for the shaft 16 and the shaft longitudinal axis 50 is as small as possible. In the preferred embodiment, the offset 65 between the shaft pivot axis 58 and the shaft longitudinal axis 50 is only about 1/20^{th} of an inch. Having an offset 65 between the shaft pivot axis 58 and the shaft longitudinal axis 50 of less than ½ inch or so provides a more balanced feel to the surgeon during pivoting of the retractor shaft 16, because the retractor shaft 16 seems to pivot rather than swing in an arc about the shaft pivot axis 58. While the offset 65 could be eliminated by either making a larger stanchion head 42, by selecting a stronger material for the stanchion head 42 and making the side stanchion 46 smaller, or by positioning the stanchion head 42 (and particularly the side stanchion 46) further from the pivot axis 58, the preferred design is very compact and tight, and the 1/20^{th} of an inch size of the offset 65 is negligible to the perception of most users.

In most surgical procedures and as depicted in the figures (particularly FIG. 5), pivoting of the shaft 16 will take the shaft 16 through a shaft travel path which, in the preferred embodiment, is a substantially horizontal plane. The handle movement direction (i.e., the surface defined by movement of the grasping axis 61 during tightening/loosening pivoting of the handle 24) is preferably at a substantial angle to the shaft travel path, such as perpendicular to the horizontal shaft travel plane.

As best shown in FIG. 3, the preferred rotatable attachment has a frustroconical bottom flange 60 formed integrally with the side stanchion 46. The frustroconical bottom flange 60 mates with a frustroconical recess 62 in a bore 64 within the upper leg portion 32 of the frame clamp 26. The frustroconical nature of this mating relationship permits pivoting of the shaft clamp 28 relative to the frame clamp 26 so long as the handle 24 is in the loosened position, but frictionally prevents pivoting of the shaft clamp 28 relative to the frame clamp 26 once pressure is applied by the handle 24 in the tightened position.

The handle 24 is keyed to the shaft clamp 28 so the handle 24 moves with the shaft clamp 28 and controls the pivoting location of the shaft clamp 28 about the vertical axis 58. The preferred keying structure is through the tightening pin 52, best shown in FIG. 3. A proximal end of the tightening pin 52 includes a non-circular yoke 66, and the central lobe 41 of the cam 38 resides within the yoke 66. A non-circular opening (not shown) in the bottom of the stanchion head 42 mates with and receives the non-circular yoke 66. The preferred non-circular yoke 66 has flats 70 to drive this rotational coupling. When the handle 24 is pivoted about the vertical axis 58, the cam 38 pivots as part of the handle 24, causing both the non-circular yoke 66 and the keyed stanchion head 42 to also pivot. After the clamp 22 is on the support frame 18 and while the handle 24 is still in the loosened position, the surgeon can easily select and adjust the orientation of the shaft clamp 28 in two ways: either by grasping the handle 24 and pivoting the handle 24 about the pivot axis 58, or by placing the retractor shaft 16 in the shaft clamp opening 48 and lightly pivoting the retractor shaft 16 about the pivot axis 58. Either way, the handle 24 always stays oriented in a vertical plane in line with the retractor shaft 16. If desired, the handle plane (defined by the movement of the grasping axis 61 during tightening/loosening pivoting of the handle 24) could alternatively be offset somewhat from the retractor shaft axis 50, preferably remaining at least parallel to the retractor shaft axis 50 if not aligned with the retractor shaft axis 50.

The preferred clamp 22 permits pivoting of the shaft 16 relative to the support frame 18 through angles θ1 and θ2 (best shown in FIG. 5) before the handle 24 interferes with the frame clamp 26 on either side. These large pivoting angles θ1 and θ2 permit great flexibility for the surgeon to determine the best angle for placement of the shaft 16. In the preferred embodiment, the counter-clockwise (from above) pivoting angle θ1 is about 45°, which will accommodate the desired angle of securement for the shaft 16 in the vast majority of applications. This high pivoting angle θ1 is achieved by the horizontal offset 53 together with the shape of the arm portion 59 of the handle 24, which avoids contact between the handle 24 and either the shaft 16 or the frame clamp 26 throughout the complete throw of the handle 24 between the loosened position and the tightened position.

The clockwise pivoting angle θ2 is even greater, and in the preferred embodiment extends about 90° before the proximal end of the handle 24 (the end of the handle 24 beyond the cam 38) interferes with the frame clamp 26. If desired, the length of the proximal end of the handle 24 could be made shorter or the offset 63 increased slightly to permit an even greater clockwise pivoting angle θ2. With the full pivoting angle θ1+θ2 being greater than 180°, any desired angle of securement is possible. For instance, if the surgeon desires to secure the shaft 16 at a 60° counterclockwise angle to the frame 18, the handle 24 could be rotated 120° clockwise and the shaft could be snapped into the shaft clamp 28 in a “backwards” orientation, with the handle 24 tightening toward the surgical arena.

To provide the desired base position for the outer cam lobes 39, the bearing surface between the cam lobes 39 and the lower leg 34 of the frame clamp 26 is provided by a variable height assembly with an adjustable base height, which includes a C-bearing 68, a plunger base 74, a spring 84 and an adjustment plug 82. The plunger base 74 and C-bearing 68 ride on the spring 84, which maintains a loosened compressive force (typically only a few pounds) biasing the assembly upwards. Transverse insertion of a properly-sized retractor shaft 16 into the shaft clamp 28 moves the pin 52 slightly downward, which in turn moves the C-bearing 68 and plunger base 74 slightly downward, against this spring force. During tightening of the clamp 22, first the cam action absorbs the spring deflection until the spring 84 bottoms out. After the spring 84 bottoms out, the remainder of the cam action causes a force loop which: a) forces the pin 52 upward to clamp the shaft 16 against the top stanchion 44, transferring the cam force through the shaft 16 to the stanchion 42, which in turn b) forces the bottom flange 60 upward to clamp the shaft clamp 28 against rotation against the recess 62 of the upper leg portion 32, transferring the cam force to the upper leg portion 32, which in turn c) forces the frame clamp 26 closed by bending at the fulcrum portion 30, to clamp the frame 18 against the lower leg portion 34. The C-bearing 68 follows the outer cam lobes 39, in an arc relative to the yoke 66 and frame clamp 26, during the entire throw of the handle 24.

In the preferred embodiment, the yoke 66 and the C-bearing 68, which bear against the cam 38 during tightening and loosening of the clamp 22, are formed of a strong bearing-grade metal, such as NITRONIC 60 super alloy. The remaining components may be formed of an appropriately strong sterilizable metal, such as surgical stainless steel.

During assembly of the preferred clamp 22 as best understood with reference to FIG. 3, the stanchion head 42 is inserted into the bore 64 of the upper leg portion 32 from below. An opening 76 in the lower leg portion 34 may be used for access to assist in machining of the upper leg portion 32 and to assist in placement of the stanchion head 42 into the bore 64 from below. An annular groove 80 on the stanchion head 42 is positioned above an upper surface of the upper leg portion 32. Once assembled in position, the stanchion head 42 is then loosely secured to the frame clamp 26 with a snap ring 78. The snap ring 78 is disposed within the annular groove 80 and rotatably holds the stanchion head 42 within the upper leg portion 32 of the frame clamp 26. The pin 52 including the non-circular yoke 66 is then positioned into its keyed opening 54 in the stanchion head 42.

The plunger base 74 is positioned into the lower leg portion 34 from above the lower leg portion 34. The handle 24 is then assembled in place from the side. The handle 24 is first positioned in the C-bearing 68, and retained in position in the C-bearing 68 with two captivation pins 72. Side ears 77 on the cam 38 maintain the cam 38 centered side to side in the C-bearing 68. Once in position, the C-bearing 68 provides stops which limit the throw of the cam 38 in both loosening and tightening to the desired 95° throw angle φ. The keyed yoke 66 is placed over the central lobe 41 of the cam 38 as the handle 24 and C-bearing 68 are slid in from the side. Side ears 77 may include flats 75 so side ears 77 do not interfere with the upper arm 32 in the loosened position.

Assembly is completed by placing a spring 84 and screwing an adjustment plug 82 in from below to bias the plunger base 74 upward. Spring 84 preferably has a low spring constant (such as 24 pounds per inch), so it will be fully compressed with a relatively small compression force (such as 4 pounds). The elevation of the adjustment plug 82 is selected by screwing an appropriate amount to provide the desired loosened and tightened (with spring 84 fully compressed) spacing for the clamp 22. For instance, the elevation of the adjustment plug 82 may be set such that a throw force on the handle 24 of 20 pounds will complete the tightening action about an appropriately sized shaft 16 and frame 18. Once the desired elevation for the adjustment plug 82 is achieved, the adjustment plug 82 is set at this elevation by upsetting the threads of the adjustment plug 82 by using an orbital riveting machine through the holes in the bottom arm 34.

Because the final seated position of the adjustment plug 82 is not set until after all the component parts are fully manufactured and assembled, the tightening force on the handle 24 is not changed by differing dimensions of the component parts within tolerance. That is, all the clamps 22 manufactured can be set to have the same tightening force, even if, for instance, the cam 38 on one clamp 22 is a mil or two larger than the cam 38 of a different clamp 22. The spring 84 places a vertically oriented force on the assembly and, together with proper tightening of the adjustment plug 82, prevents any separation or rattling of parts which might otherwise occur if the dimensional tolerances on any of these parts are not strictly met.

The preferred clamp 22 accordingly permits a loosened attachment to both the support frame 18 and the retractor shaft 16 which has five degrees of freedom: the clamp 22 can be slid longitudinally on the support frame 18; the clamp 22 can be rotated about the longitudinal axis 40 of the support frame attachment portion; the shaft 16 can be pivoted about the vertical axis 58; the shaft 16 can be slid longitudinally in the shaft clamp 28; and the shaft 16 can be rotated about the longitudinal axis 50 of the shaft attachment portion. When the handle 24 is “thrown” or pivoted from the loosened position to its tightened position, all five of these degrees of freedom are secured. During tightening, both the shaft opening and the frame opening dimensions are slightly decreased to eliminate any rotation or translation of the shaft 16 and frame 18 relative to the clamp 22. At the same time, the frictional engagement of the mating frustroconical surfaces 60, 62 after tightening prevent further pivoting of the shaft clamp 28 relative to the frame clamp 26.

The linkage between the handle 24 and the frame clamp 26 and the shaft clamp 28 is fully operated between the fully loosened position and the fully tightened position by a pivoting of the handle 24 through a tightening throw range φ shown in FIG. 4. In contrast to prior art devices, the entire tightening range φ for the clamp 22 is targeted to conform to the size and motion appropriate for a surgeon's hand. To tighten the clamp 22, the surgeon need only grasp the bottom biasing surface 86 (height shown in FIG. 4) of the shaft 16 with the surgeon's fingers and the grip portion 57 of the handle 24 with the surgeon's thumb on the same hand, and squeeze similar to operating a scissors. This scissors squeezing motion is very intuitive, as students have been taught to use scissors since kindergarten. Thus, the top of the grasping portion 57 of the handle 24 is always aligned with and is generally facing away from the bottom biasing surface 86 of the shaft 16. In particular, the grasping surface 57 of the handle 24 should remain within about six inches or less from the bottom biasing surface 86 of the shaft 16. The handle 24 should move an entire distance of about five inches or less. To enable the scissors action, the tightening throw should proceed through an angle φ of 120° or less. In the tightened position, the grasping surface 84 of the handle 24 is positioned a distance d of from ½ to three inches from the bottom biasing surface 86, which enables a strong single handed grasping force to the fully tightened position. In the preferred embodiment, the tightened squeeze distance d is about 1½ inches. The preferred handle 24 extends for a length of about 3 inches, and pivots through a tightening throw range φ of about 95°.

In the fully tightened position, the grasping portion 57 of the handle 24 extends at a slight angle α to the shaft axis 50. This grasping angle σ, though not critical, assists in application of a greater squeeze force by the surgeon's normal grip, and also assists in providing clearance for releasing the clamp 22. In the preferred embodiment, the tightened grasping angle σ is about 5°. The bottom side of the grasping portion 57 of the handle 24 provides a spacing 96 of about ¾ of an inch over the top 88 of the shaft 16 for loosening access to the handle 24.

At this size, amount of pivoting and location of the handle pivot axis 43, the grasping surface 57 of the handle 24 is 4 inches away from the bottom biasing surface 86 of the shaft 16 while in the loosened position, and is about ½ inch away from the bottom biasing surface 86 of the shaft 16 when in the tightened position. The handle/shaft combination thus act in conjunction to ergonomically fit the grasp of most surgeons' hands for a single handed, intuitive tightening operation. The surgeon is most commonly standing roughly in line and behind the retractor shaft 16. Just as when cutting a wide cloth a seamstress will lean over a flatly laid cloth and cut away from his or her body, so too the surgeon tightens the clamp 22 with a natural “cutting” orientation, fingers down and thumb up, similar to a handshake position. While the clamp 22 can be readily tightened with a wide variety of single handed orientations, the most common hand orientation naturally coincides with the most common clamp orientation and strongest grip orientation relative to the person tightening the clamp 22.

As best shown in FIGS. 5-7, the arm portion 59 of the handle 24 in the preferred clamp 22 is offset with offset 53 relative to the shaft axis 50, primarily to provide sufficient clearance to maximize the pivot angles θ1 and θ2 and to provide loosened and tightened clearance 55 between the shaft 16 and the handle 24.

As commonly desired, the clamp 22 is capable of being used with a vertical plane containing the shaft axis 50 being oriented perpendicular to the support frame axis 40. For instance, with the shaft/handle of the clamp 22 as shown in the solid lined plan view of FIG. 5, the entire clamp 22 is generally balanced and largely symmetrical about a bisecting vertical plane. When the tightening force is applied in this most common position, the plane defined by the grasping portion 57 tends to orient the tightening force so as to provide the maximum rotational moment on the cam 38 while minimizing any twisting moment of the clamp 22 off the support frame 18. By having a largely balanced, most-common position, the clamp 22 is less likely to twist off the support frame 18 during tightening.

Whether in the tightened position or in the loosened position, the grip portion 57 of the handle 24 always resides vertically above the shaft 16. The shaft 16 is accordingly always in place below the handle 24 for the surgeon's hand to provide equal and opposite squeeze forces on the clamp 22 and hold the clamp 22 from rotating about the support frame 18 during tightening. Orienting the grip portion 57 of the handle 24 and the shaft 16 always in alignment is particularly important when the shaft 16 is at a non-perpendicular angle to the support frame 18, so the tightening forces can be carefully and easily controlled, via single-handed operation, without causing the frame clamp 26 to twist off the support frame 18. To provide this desired orientation of the grip portion 57 of the handle 24, the arm portion 59 of the handle 24 has a vertical S-curve offset 90 (shown in FIG. 4) and a horizontal S-curve to provide the offset 53 (shown in FIG. 7). In the preferred embodiment, the horizontal offset 53 is about 0.8 inches, and the vertical offset 90 from the handle pivot axis 43 to the top of the grip portion 57 in the tightened position is about 2 inches.

The “over the top and downward” throw of the handle 24 of the preferred clamp 22 assists in avoiding any interference between the handle 24 and the patient's body or garments. Even if the support frame 18 is positioned very close or in contact with a patient's body or garments, the handle 24 will be accessible from above for its complete throw without interference in any way from the support frame 18 or the patient. In the loosened position (shown in FIG. 4 in dashed lines), the preferred handle 24 projects forward from the frame axis 40 by only a spacing 102 of about 1¼ inches, for a total throw of about 5¼ inches.

In the tightened position of the preferred clamp 22 with the most common orientation of the handle/shaft (in the six o'clock position shown in solid lines in FIG. 5), the grasping portion 57 of the handle is disposed outward from the surgical arena to provide a very low profile clamping arrangement. This is best understood with reference to FIG. 4, wherein:

a) the distal end of the handle 24 has a tightened elevation 92 over the shaft 16 of only about 1 inch, and this highest elevation is spaced distally outward from the frame by a spacing 100 of about 4 inches;

b) the stanchion head 42 projects an elevation 94 of less than ¼ of an inch over the shaft 16, such as a profile elevation 94 of about 0.1 inches; and

c) the bottom surface 86 of the shaft 16 is a distance 98 of only about ¾ of an inch over the support frame 18.

With this configuration, the tightened clamp 22 virtually never interferes with the surgeon's line of sight or access to the surgical arena.

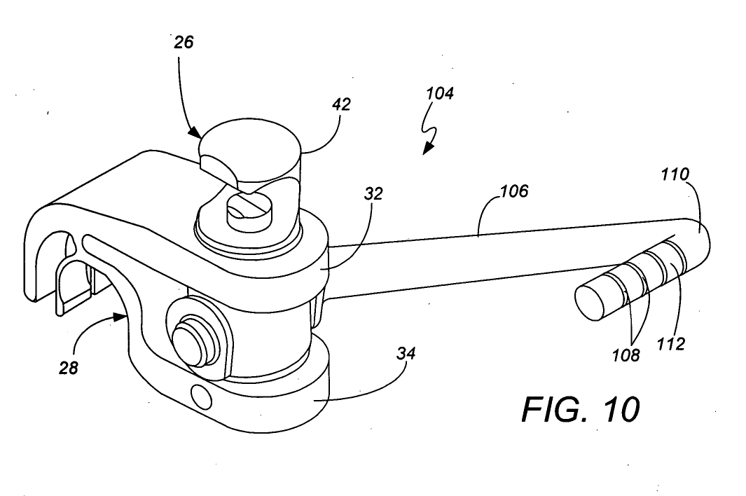

FIG. 10 depicts an alternative embodiment of a clamp 104 in accordance with many aspects of the present invention. In clamp 104, the handle 106 is generally formed by machining the cam (not shown) and handle ornamentation 108 into rod stock, and then adding to simple 90° bends 110 (one hidden from view) to the rod stock. This handle 106 is simpler to make than the handle 24 of the first embodiment, and provides many of the advantages of the invention. However, the gripping portion 112 of the handle 106 is perpendicular (rather than aligned) with the shaft axis, and the “scissors action” for tightening of the clamp 104 is not so intuitive. Also, with the gripping portion 112 at an angle relative to the shaft axis, aligned placement of the tightening force (so as to place no moment relative to the shaft) is not so intuitive. However, cost savings associated with the simpler handle 106, together with achieving many benefits of this invention, make this clamp 104 a viable lower cost alternative.

Thus it can be seen that the location and orientation of the handle 24 relative to the frame clamp 26 and relative to the shaft clamp 28 provide many advantages during the surgical procedure which are not provided by prior art clamps. While many linkage mechanisms can be used to translate tightening and loosening forces between the handle 24 and the frame clamp 26 and shaft clamp 28, the preferred linkage is cost effective, simple to manufacture and provides a very robust and easy to use clamp in an elegant design. The preferred linkage also uses the same cam 38 to transmit both the tightening force and a pivoting moment about axis 58 which keeps the handle 24 aligned with the shaft 16.

Although the present invention has been described with reference to preferred embodiments, workers skilled in the art will recognize that changes may be made in form and detail without departing from the spirit and scope of the invention. For instance, while the preferred embodiment includes two clamping members, certain aspects of the invention could be practiced with a single clamping member, such as integral with and/or permanently attached to either the support frame 18 or the retractor shaft 16. While a particular linkage is described which enables the handle 24 to simultaneously control both clamping members, it is recognized that many other types of linkages could be used while still obtaining the handle orientation benefits of the present invention.

1. A surgical clamp comprising:
a first clamp member having a frame rod opening for receiving a support frame rod, the frame rod opening being accessible for transverse application onto the first rod, the frame rod opening defining a frame rod opening axis;
a second clamp member having a retractor rod opening for receiving a retractor rod, the retractor rod opening being accessible for transverse application of the retractor rod into the retractor rod opening at an orientation wherein the retractor rod extends in a retractor rod plane above the frame rod, with the retractor rod plane being defined parallel to the frame rod opening axis and the retractor rod opening defining an axis within the retractor rod plane, the second opening changing dimensions to receive the retractor rod in a loosened second clamp position and in a tightened second clamp position, the second clamp member being attached to the first clamp member;
a handle having a handle linkage for moving the second clamp between its loosened second clamp position and its tightened second clamp position, the handle linkage coupled to the second clamp at a position below the retractor rod plane, the handle having a grip portion which is above the retractor rod plane in both the loosened second clamp position and in the tightened second clamp position.

2. The surgical clamp of claim 1, wherein the second clamp member and the handle are both on the same side of the first clamp member.

3. The surgical clamp of claim 1, wherein the grip portion of the handle pivots in a handle plane, wherein the handle plane always substantially contains or is parallel to the retractor rod opening axis.

4. The surgical clamp of claim 1, wherein pivoting of the handle moves the first clamp between its loosened first clamp position and its tightened first clamp position simultaneously with moving the second clamp between its loosened second clamp position and its tightened second clamp position.

5. The surgical clamp of claim 1, wherein when in the loosened second clamp position the second clamp member is pivotable relative to the first clamp member about a pivot axis.

6. The surgical clamp of claim 5, wherein the handle pivots about the pivot axis together with the second clamp member.

7. The surgical clamp of claim 6, wherein the handle moves the first clamp between a loosened first clamp position and a tightened first clamp position and tightens or loosens the pivotable connection between the first clamp member and the second clamp member simultaneously with moving the second clamp between its loosened second clamp position and its tightened second clamp position

8. A surgical clamp comprising:
a first clamp member having a first rod opening for receiving a first rod, the first opening changing dimensions to receive the first rod in a loosened first clamp position and in a tightened first clamp position;
a second clamp member having a second rod opening for receiving a second rod, the second opening changing dimensions to receive the retractor rod in a loosened second clamp position and in a tightened second clamp position, the second clamp member being attached to the first clamp member;
a handle having a handle linkage both for moving the first clamp between its loosened first clamp position and its tightened first clamp position and for moving the second clamp between its loosened second clamp position and its tightened second clamp position, the handle linkage comprising:
a cam member biasing off a bearing surface about a cam center plane, the cam member pivotally rotating about a cam axis;
a shaft portion extending along the cam axis off a single side of the cam member for applying torque to rotate the cam member; and
a handle extension extending to grip portion, the handle extension being bent such that the grip portion extends through or within the cam center plane.

9. The surgical clamp of claim 8, wherein the first rod opening defines a first rod opening axis and the second rod opening defines a second rod opening axis, and, with the surgical clamp oriented such that both the first rod opening axis and the second rod opening axis are horizontal and with the handle in the tightened second clamp position, the handle has a vertical S-curve offset and a horizontal S-curve offset.

10. The surgical clamp of claim 8, wherein the first rod opening defines a first rod axis, wherein the first rod opening is open for receiving the first rod in a direction transverse to the first rod axis, wherein the second opening defines a second rod axis, and wherein the second opening is open for receiving the second rod in a direction transverse to the second rod axis.

11. The surgical clamp of claim 8, wherein when in the loosened position the second clamp member is pivotable relative to the first clamp member about a pivot axis.

12. The surgical clamp of claim 11, wherein the handle pivots about the pivot axis together with the second clamp member.

13. The surgical clamp of claim 12, wherein the handle tightens or loosens the pivotable connection between the first clamp member and the second clamp member simultaneously with moving the first and second clamps between their tightened and loosened positions.

14. A surgical clamp comprising:
a first clamp member having a first rod opening for receiving a first rod, the first rod opening defining a first rod opening axis, the first opening changing dimensions to receive the first rod in a loosened first clamp position and in a tightened first clamp position;
a second clamp member having a second rod opening for receiving a second rod, the second rod opening defining a second rod opening axis, the second opening changing dimensions to receive the second rod in a loosened second clamp position and in a tightened second clamp position, the second clamp member being attached to the first clamp member;
a handle both for moving the first clamp between its loosened first clamp position and its tightened first clamp position and for moving the second clamp between its loosened second clamp position and its tightened second clamp position, the handle having a grip portion defining a plane of tightening force application, wherein a tightening force applied to the grip portion normal to the plane of tightening force application intersects the second clamp opening axis.

15. The surgical clamp of claim 14, wherein, with the handle in the tightened position, the plane of tightening force application is parallel to the second rod opening axis.

16. The surgical clamp of claim 14, wherein the grip portion contains a central hole therethrough, normal to the plane of tightening force application.

17. The surgical clamp of claim 14, wherein when in the loosened position the second clamp member is pivotable relative to the first clamp member about a pivot axis.

18. The surgical clamp of claim 17, wherein the handle pivots about the pivot axis together with the second clamp member.

19. The surgical clamp of claim 18, wherein the handle tightens or loosens the pivotable connection between the first clamp member and the second clamp member simultaneously with moving the first and second clamps between their tightened and loosened positions.
